(19) United States
(12) Patent Application Publication
(10) Pub. No.: US 2022/0313349 A1
(43) Pub. Date: Oct. 6, 2022

(54) SYSTEM AND METHOD OF DITHERING TO MAINTAIN GRASP FORCE

(71) Applicant: INTUITIVE SURGICAL OPERATIONS, INC., Sunnyvale, CA (US)

(72) Inventors: Adrit LATH, Palo Alto, CA (US); Brian D. SOE, Oakland, CA (US)

(21) Appl. No.: 17/642,205

(22) PCT Filed: Sep. 10, 2020
(86) PCT No.: PCT/US2020/050132

§ 371 Date: Mar. 10, 2022

Related U.S. Application Data
(60) Provisional application No. 62/899,085, filed on Sep. 11, 2019.

(51) Int. Cl. A61B 18/14; A61B 34/30; A61B 34/00

(52) U.S. Cl. CPC A61B 18/1442; A61B 34/30; A61B 34/71; A61B 2090/066

(57) ABSTRACT

Systems and methods of dithering to maintain grasp force include a computer-assisted device. The computer-assisted device includes an instrument having a first jaw and a second jaw configured to grasp a material, one or more actuators configured to actuate the first and second jaws to apply force to the grasped material, and a controller coupled to the one or more actuators. The controller is configured to determine that actuation of the one or more actuators should be dithered and in response to the determination, dither one or more control signals to the one or more actuators so as to cause variations in a force or torque applied by the one or more actuators. In some embodiments, the one or more control signals correspond to a force setpoint, a torque setpoint, a current setpoint, or a position setpoint for the one or more actuators.

### RELATED APPLICATIONS

This application claims the benefit to U.S. Provisional Application No. 62/899,085, filed Sep. 11, 2019 and entitled “System and Method of Dithering to Maintain Grasp Force,” which is incorporated by reference herein.

### TECHNICAL FIELD

The present disclosure relates generally to operation of devices with repositionable arms and instruments and more particularly to operation of a grasping instrument using dithering to maintain consistent grasp force and prevent excessive force generation.

### BACKGROUND

More and more devices are being replaced with computer-assisted electronic devices. This is especially true in industrial, entertainment, educational, and other settings. As a medical example, the hospitals of today with large arrays of electronic devices being found in operating rooms, interventional suites, intensive care wards, emergency rooms, and/or the like. For example, glass and mercury thermometers are being replaced with electronic thermometers, intravenous drip lines now include electronic monitors and flow regulators, and traditional hand-held surgical and other medical instruments are being replaced by computer-assisted medical devices.

These computer-assisted devices are useful for performing operations and/or procedures on materials, such as the bodily tissue of a patient, that are located in a workspace. With many computer-assisted devices, an operator (such as surgeon in a medical example) may remotely teleoperate a computer-assisted device using one or more controls on an operator console. As the operator manipulates the various controls at the operator console, the commands are relayed from the operator console to a device, in or near the workspace, to which one or more end effectors and/or instruments are mounted. In this way, the operator is able to perform one or more procedures on a material or object in the workspace using the end effectors and/or instruments. Depending upon the desired procedure and/or the instruments in use, the desired procedure may be performed partially or wholly under control of the operator using teleoperation and/or under semi-autonomous control where the instrument may perform or alter a sequence of operations based on one or more activation actions by the operator.

Instruments of different design and/or configuration may be used to perform different tasks, procedures, and/or functions so as to be allow the operator to perform any of a variety of procedures. Computer-assisted instruments, whether actuated manually, teleoperatively, and/or semi-autonomously, may be used in a variety of operations and/or procedures and may have various configurations. Many such instruments include an end effector mounted at a distal end of a shaft that may be mounted to the distal end of a repositionable arm. In many operational scenarios, the shaft may be configured to be inserted (e.g., laparoscopically, thoracoscopically, and/or the like) through an opening (e.g., a body wall incision, a natural orifice, an access port, and/or the like) to reach a remote site within the workspace. In some instruments, an articulated wrist mechanism may be mounted to the distal end of the instrument's shaft to support the end effector with the articulated wrist providing the ability to alter an orientation of the end effector relative to a longitudinal axis of the shaft. Examples of such instruments include, but are not limited to, cauterizing, ablating, suturing, cutting, stapling, fusing, sealing, etc., and/or combinations thereof. Accordingly, instruments can include a variety of components and/or combinations of components to perform these procedures.

When access to the workspace is limited, the size and/or strength of the instruments may be limited. As a medical example, when the computer-assisted device is being used to perform a minimally invasive surgical procedure, the instruments may have to fit through a bodily orifice and/or a body wall incision that is kept as small as possible to reduce the impact on the patient. In another example, when the workspace is contained (e.g., it is hazardous, climate controlled, sterile, and/or the like), access to the workspace may be controlled through one or more orifices that may be of reduced size. Consistent with the goals of accessing a workspace having limited access, the size of the end effector is typically kept as small as possible while still allowing it to perform its intended task. One approach to keeping the size of the end effector small is to accomplish actuation of the end effector through the use of one or more inputs at a proximal end of the surgical instrument, which is typically located externally to the patient. Various gears, levers, pulleys, cables, rods, bands, and/or the like, may then be used to transmit actions from the one or more inputs along the shaft of the instrument and to actuate the end effector. For example, a transmission mechanism at the proximal end of the instrument interfaces with various actuators, such as motors, solenoids, servos, hydraulics, pneumatics, and/or the like provided on a repositionable arm of the computer-assisted device located in or near the workspace. The actuators typically receive control signals through a master controller and provide input in the form of force and/or torque at the proximal end of the transmission mechanism, which the various gears, levers, pulleys, cables, rods, bands, and/or the like ultimately transmit to actuate the end effector at the distal end of the transmission mechanism.

Given the smaller size of the instrument and the use of the transmission mechanism it may be desirable to operate the instrument using as small a force and/or torque that is sufficient to perform the desired procedure. This helps reduce the size of the instrument, reduce the likelihood of exerted more force and/or torque than is necessary, reduces wear and tear on the instrument, and/or the like.

Accordingly, improved methods and systems for the operation of instruments while appropriately maintaining the amount of an exerted force and/or torque are desirable.

### SUMMARY

Consistent with some embodiments, a computer-assisted device includes an instrument having a first jaw and a second jaw configured to grasp a material, one or more actuators configured to actuate the first and second jaws to apply force to the grasped material, and a controller coupled to the one or more actuators. The controller is configured to determine that actuation of the one or more actuators should be dithered and in response to the determination, dither one or more control signals to the one or more actuators so as to cause variations in a force or torque applied by the one or more actuators.

Consistent with some embodiments, a method includes determining, by a controller of a computer-assisted device, that actuation of one or more actuators of the computer-assisted device should be dithered, the one or more actuators usable to actuate a first jaw and a second jaw of an instrument to grasp a material. The method further includes, in response to the determining, dithering, by the controller, one or more control signals to the one or more actuators so as to cause variations in a force or torque applied by the one or more actuators.

Consistent with some embodiments, a non-transitory machine-readable medium comprising a plurality of machine-readable instructions which when executed by one or more processors associated with a computer-assisted medical device are adapted to cause the one or more processors to perform any of the methods described herein.

### BRIEF DESCRIPTION OF THE DRAWINGS

FIG. 1 is a simplified diagram of a computer-assisted system according to some embodiments.

FIG. 2 is a simplified diagram showing an instrument according to some embodiments.

FIG. 3 is a simplified perspective diagram of the distal end of the instrument of FIG. 2 according to some embodiments.

FIG. 4 is a simplified diagram of a method of operating an instrument according to some embodiments.

In the figures, elements having the same designations have the same or similar functions.

### DETAILED DESCRIPTION

This description and the accompanying drawings that illustrate inventive aspects, embodiments, implementations, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, this description's terminology is not intended to limit the invention. For example, spatially relative terms-such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, or module may, whenever practical, be included in other embodiments, implementations, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an implementation using a surgical system, such as the da Vinci® Surgical System commercialized by Intuitive Surgical, Inc. of Sunnyvale, Calif. Knowledgeable persons will understand, however, that inventive aspects disclosed herein may be embodied and implemented in various ways, including robotic and, if applicable, non-robotic embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects.

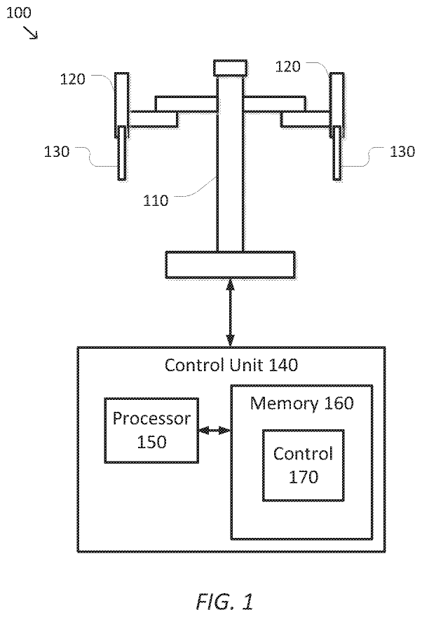

FIG. 1 is a simplified diagram of a computer-assisted system 100 according to some embodiments. As shown in FIG. 1, computer-assisted system 100 includes a computer-assisted device 110 with one or more movable or repositionable arms 120. Each of the one or more repositionable arms 120 may support one or more instruments 130. In some examples, computer-assisted device 110 may be consistent with a computer-assisted surgical device. The one or more repositionable arms 120 may each provide support for instruments 130 such as surgical instruments, imaging devices, and/or the like. In some examples, the instruments 130 may include end effectors that are capable of, but are not limited to, performing, gripping, retracting, cauterizing, ablating, suturing, cutting, stapling, fusing, sealing, etc., and/or combinations thereof.

Computer-assisted device 110 may further be coupled to an operator workstation (not shown), which may include one or more master controls for operating the computer-assisted device 110, the one or more repositionable arms 120, and/or the instruments 130. In some examples, the one or more master controls may include master manipulators, levers, pedals, switches, keys, knobs, triggers, and/or the like. In some embodiments, computer-assisted device 110 and the operator workstation may correspond to a da Vinci® Surgical System commercialized by Intuitive Surgical, Inc. of Sunnyvale, Calif. In some embodiments, computer-assisted surgical devices with other configurations, fewer or more repositionable arms, and/or the like may be used with computer-assisted system 100.

Computer-assisted device 110 is coupled to a control unit 140 via an interface. The interface may include one or more cables, fibers, connectors, and/or buses and may further include one or more networks with one or more network switching and/or routing devices. Control unit 140 includes a processor 150 coupled to memory 160. Operation of control unit 140 is controlled by processor 150. And although control unit 140 is shown with only one processor 150, it is understood that processor 150 may be representative of one or more central processing units, multi-core processors, microprocessors, microcontrollers, digital signal processors, field programmable gate arrays (FPGAs), application specific integrated circuits (ASICs), tensor processing units (TPUs), and/or the like in control unit 140. Control unit 140 may be implemented as a stand-alone subsystem and/or board added to a computing device or as a virtual machine. In some embodiments, control unit 140 may be included as part of the operator workstation and/or operated separately from, but in coordination with the operator workstation.

Memory 160 may be used to store software executed by control unit 140 and/or one or more data structures used during operation of control unit 140. Memory 160 may include one or more types of machine readable media. Some common forms of machine readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

As shown in FIG. 1, memory 160 includes a control module 170 that may be used to support autonomous, semiautonomous, and/or teleoperated control of computer-assisted device 110. Control module 170 may include one or more application programming interfaces (APIs) for receiving position, motion, force, torque, and/or other sensor information from computer-assisted device 110, repositionable arms 120, and/or instruments 130, exchanging position, motion, force, torque, and/or collision avoidance information with other control units regarding other devices, and/or planning and/or assisting in the planning of motion for computer-assisted device 110, repositionable arms 120, and/or instruments 130. In some examples, control module 170 may further support autonomous, semiautonomous, and/or teleoperated control of the instruments 130 during a procedure, such as a surgical procedure. And although control module 170 is depicted as a software application, control module 170 may be implemented using hardware, software, and/or a combination of hardware and software.

In some medical embodiments, computer-assisted system 100 may be found in an operating room and/or an interventional suite. And although computer-assisted system 100 includes only one computer-assisted device 110 with two repositionable arms 120 and corresponding instruments 130, one of ordinary skill would understand that computer-assisted system 100 may include any number of computer-assisted devices with repositionable arms and/or instruments of similar and/or different in design from computer-assisted device 110. In some examples, each of the computer-assisted devices may include fewer or more repositionable arms and/or instruments.

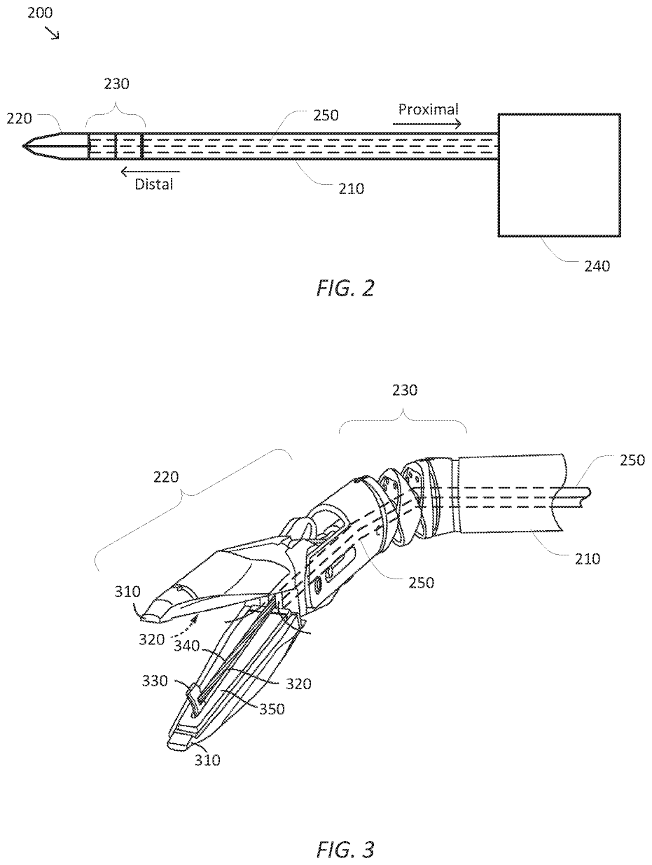

FIG. 2 is a simplified diagram showing an instrument 200 according to some embodiments. In some embodiments, instrument 200 may be consistent with any of the instruments 130 of FIG. 1. The directions “proximal” and “distal” as depicted in FIG. 2 and as used herein help describe the relative orientation and location of components of instrument 200. Distal generally refers to elements in a direction further along a kinematic chain from a base of a computer-assisted device, such as computer-assisted device 110, and/or or closest to the workspace in the intended operational use of the instrument 200. Proximal generally refers to elements in a direction closer along a kinematic chain toward the base of the computer-assisted device and/or one of the repositionable arms of the computer-assisted device.

As shown in FIG. 2, instrument 200 includes an elongated shaft 210 used to couple an end effector 220 located at a distal end of shaft 210 to where the instrument 200 is mounted to a repositionable arm and/or a computer-assisted device at a proximal end of shaft 210. Depending upon the particular procedure for which the instrument 200 is being used, shaft 210 may be inserted through an opening (e.g., a body wall incision, a natural orifice, a workspace port, and/or the like) in order to place end effector 220 in proximity to a remote work site located within the workspace (e.g., an interior anatomy of a patient in a medical example). As further shown in FIG. 2, end effector 220 is generally consistent with a two-jawed gripper-style end effector, which in some embodiments may further include a cutting and/or a fusing or sealing mechanism as is described in further detail below with respect to FIG. 3. However, one of ordinary skill would understand that different instruments 200 with different end effectors 220 are possible and may be consistent with the embodiments of instrument 200 as described elsewhere herein.

An instrument, such as instrument 200 with end effector 220 typically relies on multiple degrees of freedom (DOFs) during its operation. Depending upon the configuration of instrument 200 and the repositionable arm and/or computer-assisted device to which it is mounted, various DOFs that may be used to position, orient, and/or operate end effector 220 are possible. In some examples, shaft 210 may be inserted in a distal direction and/or retreated in a proximal direction to provide an insertion DOF that may be used to control how deep within the workspace end effector 220 is placed. In some examples, shaft 210 may be able rotate about its longitudinal axis to provide a roll DOF that may be used to rotate end effector 220. In some examples, additional flexibility in the position and/or orientation of end effector 220 may be provided by an optional articulated wrist 230 that is used to couple end effector 220 to the distal end of shaft 210. In some examples, articulated wrist 230 may include one or more rotational joints, such as one or more roll, pitch or yaw joints that may provide one or more “roll,” “pitch,” and “yaw” DOF(s), respectively, that may be used to control an orientation of end effector 220 relative to the longitudinal axis of shaft 210. In some examples, the one or more rotational joints may include a pitch and a yaw joint; a roll, a pitch, and a yaw joint, a roll, a pitch, and a roll joint; and/or the like. In some examples, end effector 220 may further include a grip DOF used to control the opening and closing of the jaws of end effector 220 and/or an activation DOF used to control the extension, retraction, and/or operation of a cutting mechanism.

Instrument 200 further includes a drive system 240 located at the proximal end of shaft 210. Drive system 240 includes one or more components for introducing forces and/or torques to instrument 200 that may be used to manipulate the various DOFs supported by instrument 200. In some examples, drive system 240 may include one or more actuators such as motors, solenoids, servos, hydraulics, pneumatics, and/or the like that are operated based on signals received from a control unit, such as control unit 140 of FIG. 1. In some examples, the signals may include one or more currents, voltages, pulse-width modulated wave forms, and/or the like. In some examples, drive system 240 may include one or more shafts, gears, pulleys, rods, bands, and/or the like which may be coupled to corresponding actuators that are part of the repositionable arm, such as any of the repositionable arms 120, to which instrument 200 is mounted. In some examples, the one or more drive inputs, such as shafts, gears, pulleys, rods, bands, and/or the like, may be used to receive forces and/or torques from the actuators and apply those forces and/or torques to adjust the various DOFs of instrument 200.

In some embodiments, the forces and/or torques generated by and/or received by drive system 240 may be transferred from drive system 240 and along shaft 210 to the various joints and/or elements of instrument 200 located distal to drive system 240 using one or more drive mechanisms 250. In some examples, the one or more drive mechanisms 250 may include one or more gears, levers, pulleys, cables, rods, bands, and/or the like. In some examples, shaft 210 is hollow and the one or more drive mechanisms 250 pass along the inside of shaft 210 from drive system 240 to the corresponding DOF in end effector 220 and/or articulated wrist 230. In some examples, each of the one or more drive mechanisms 250 may be a cable disposed inside a hollow sheath or lumen in a Bowden cable like configuration. In some examples, the cable and/or the inside of the lumen may be coated with a low-friction coating such as polytetrafluoroethylene (PTFE) and/or the like. In some examples, as the proximal end of each of the cables is pulled and/or pushed inside drive system 240, such as by wrapping and/or unwrapping the cable about a capstan or shaft, the distal end of the cable moves accordingly and applies a suitable force and/or torque to adjust one of the DOFs of end effector 220, articulated wrist 230, and/or instrument 200.

FIG. 3 is a simplified perspective diagram of the distal end of instrument 200 according to some embodiments. As shown in FIG. 3, the distal end of instrument 200 is depicted so as to show additional details of end effector 220, articulated wrist 230, and the one or more drive mechanisms 250. In more detail, end effector 220 includes opposing jaws 310 shown in an open position. Jaws 310 are configured to move between open and closed positions so that end effector 220 may be used during a procedure to grasp and release a material (e.g., tissue in a medical example) and/or other objects at the remote work site. In some examples, jaws 310 may be operated together as a single unit with both jaws 310 opening and/or closing at the same time. In some examples, jaws 310 may be opened and/or closed independently so that, for example, one jaw 310 could be held steady which the other jaw 310 may be opened and/or closed.

FIG. 3 shows that a gripping surface on an inside of each of jaws 310 includes a corresponding groove 320, which may act as a guide for an optional cutting blade 330, although the groove 320 may be omitted from one or more of jaws 310. As cutting blade 330 is extended toward the distal end of end effector 220 and/or retracted toward the proximal end of end effector 220, each of the grooves 320 may aid in the alignment and/or positioning of cutting blade 330 during a cutting operation. Extraction and/or retraction of cutting blade 330 is accomplished using a drive component 340 to which cutting blade 330 is attached. In some examples, drive component 340 pushes on cutting blade 330 to extend cutting blade 330 and pulls on cutting blade 330 to retract cutting blade 330.

In some embodiments, the gripping surface on the inside of each of jaws 310 may further include one or more optional electrodes 350. In some examples, electrodes 350 may be used to deliver electrosurgical energy to fuse and/or cut a material (e.g., tissue in a medical example) being held between jaws 310. In some examples, electrodes 360 may provide an electro-cautery, fusing, and/or sealing feature to end effector 220 so that a material may be cut and/or fused/sealed using the same instrument 200.

In some embodiments, operation of jaws 310, cutting blade 330, and/or the joints of articulated wrist 230 may be accomplished using corresponding ones of the one or more drive mechanisms 250. In some examples, when jaws 310 are operated independently, a distal end of two of the one or more drive mechanisms 250 (one for each of jaws 310) may be coupled to a respective jaw 310 so that as the corresponding one or more drive mechanisms 250 applies a pull and/or a pushing force (for example, using a cable, lead screw, and/or the like), the respective jaw 310 may be opened and/or closed. In some examples, when jaws 310 are operated together, both jaws 310 may be coupled to the distal end of the same one or more drive mechanisms 250. In some examples, drive component 340 may be coupled to a distal end of a corresponding drive mechanisms 250 so that forces and/or torques applied to the corresponding one or more of the one or more drive mechanisms 250 may be transferred to the push and/or pull motion of drive component 340. In some examples, additional one or more drive mechanisms 250 may be used to operate the roll, pitch, and/or yaw DOFs in articulated wrist 230.

According to some embodiments, the amount of grasp force exerted by jaws 310 on a grasped material may be affected by one or more of a speed with which jaws 310 are being closed, an amount of articulation in articulated wrist 230, and/or other factors. In some examples, higher amounts of articulation in articulated wrist 230 may cause an increase in an amount of grasp force exerted by jaws 310 on the grasped material (e.g., tissue in a medical example). In some examples, articulating the wrist while grasping on tissue will cause the amount of force exerted on the tissue to change. In some examples, the amount of grasp force may also be affected by the friction (e.g., static friction or stiction) in instrument 200 (e.g., in the actuators, in drive system 240, between the actuators and/or drive system 240 and the one or more drive mechanisms 250, due to articulation of articulated wrist 230, between the one or more drive mechanisms 250 and end effector 220, and/or the like) and/or between jaws 310 and/or the one or more electrodes 350 and the grasped material. In some examples, this ambiguity in grasp force is undesirable for usability, manufacturing, and controls reasons as it creates uncertainty as to whether the material is properly grasped (e.g., before other actions are performed such as cutting, stapling, energy delivery, and/or the like), may lead to excess wear and tear on instrument 200 if not properly limited, and/or the like.

According to some embodiments, several approaches may be used to address these concerns. In some examples, reducing the friction between jaws 310 and/or the one or more electrodes 350 and the grasped material by selecting appropriate surface materials, surface finish, lubrication, and/or the like may help reduce the impact of friction. In some examples, rapidly increasing grasp force to jolt the grip mechanism and “reset” friction (e.g., converting static friction to dynamic friction in the one or more drive mechanisms 250, and/or the like) may be helpful. In some examples, preventing changes in the articulation of articulated wrist 230 when sealing and cutting the gripped material using the one or more electrodes 350 may limit the impact of the articulation of articulated wrist 230. In some examples, adjusting the amount of force and/or torque exerted by one or more actuators for jaws 310 based on the amount of articulation of articulated wrist 230 may also limited the impact of the articulation of articulated wrist 230.

In some examples, reducing surface friction of jaws 310 and/or the one or more electrodes 350 provides only a limited improvement as surface friction cannot be lowered below a reasonable threshold that is needed to maintain a suitable grip on the grasped tissue by jaws 310. In some examples, preventing changes in the articulation of articulated wrist 230 negatively impacts usability. In some examples, approaches that increase the grasp force with a jolt and/or based solely on the amount of articulation in articulated wrist 230 may results in a higher than necessary grasp force, which may lead to excessive grasp forces being applied to the grasped material, undesirable wear and tear (and loss of life) for instrument 200, and/or the like.

According to some embodiments, dithering (e.g., rapidly modulating) the amount of force and/or torque applied by the one or more actuators used to control the amount of grasp force between jaws 310 and/or the one or more electrodes 350 and the grasped material may address the friction issues without resulting in undesirable increases in the grasp force. In some examples, dithering helps convert the static friction in instrument 200 (e.g., in the one or more drive mechanisms) and/or between jaws 310 and/or the one or more electrodes 350 and grasped material to a dynamic friction that allows the application of suitable grasp forces on the grasped material while avoiding high spikes in force and/or torque applied by an actuator that may cause undesirable wear and tear to instrument 200. In some examples, the dithering causes movement in instrument 200 (e.g., in the one or more drive mechanisms) that lead to variations in the amount of force applied by jaws 310 to grasp the material.

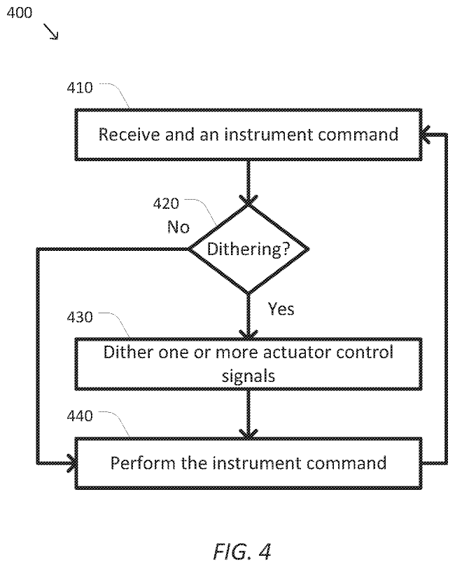

FIG. 4 is a simplified diagram of a method 400 of operating an instrument according to some embodiments. One or more of the processes 410-440 of method 400 may be implemented, at least in part, in the form of executable code stored on non-transient, tangible, machine readable media that when run by one or more processors (e.g., the processor 150 in control unit 140) may cause the one or more processors to perform one or more of the processes 410-440. In some embodiments, method 400 may be performed by a module, such as control module 170. In some embodiments, method 400 may be used to operate the instrument so that an appropriate grasp force may be maintained on a grasped material (e.g., tissue in a medical example) to perform a procedure while avoiding the exertion of excess grasp force due to articulation of an articulated wrist, friction within the instrument or between the instrument and the grasped material, and/or the like. In some examples, method 400 may be used to perform a procedure using instrument 200.

According to some embodiments, method 400 may include additional processes not shown in FIG. 4. In some embodiments, processes 410-440 of method 400 may be performed in orders other than those implied by the arrangement of FIG. 4. In some examples, processes 420 and/or 430 may be performed concurrently with process 440.

At a process 410, an instrument command is received. In some examples, the instrument command may be received from an operator, such as by using an operator console. In some examples, the operator console may be the operator console described with respect to FIG. 1. In some examples, the instrument command may be received over an interface. In some examples, the instrument command may be received via one or more API calls to a control module, such as control module 170. In some examples, the instrument command may be a command to apply cutting and/or sealing energy to a grasped material, such as might be applied by the one or more electrodes 350. In some examples, the instrument command may be a command changing an articulation of an articulated wrist, such as articulated wrist 230.

At a process 420, it is determined whether dithering should be performed. In some examples, the determination may depend on one or more of a type of the instrument command received during process 410, a current configuration of the instrument (e.g., an amount of articulation of the articulated wrist, a separation between jaws (such as jaws 310) of the instrument, an increased and/or a decrease in an amount of force to be exerted by the jaws, and/or the like), and/or the like. In some examples, when the instrument command is a command to apply cutting and/or sealing energy, dithering should be performed. In some examples, when the instrument command is to change the articulation of the articulated wrist, dithering should be performed when the instrument command results in one or more articulation conditions being present. In some examples, the one or more articulation conditions include whether the wrist is being articulated while a material is grasped between jaws 310. In some examples, the one or more articulation conditions include whether the amount of articulation of the articulated wrist exceeds a configurable angular threshold (e.g., 15-60 degrees), a change in the articulation of the articulated wrist exceeds a configurable angular change threshold (e.g., 15-60 degrees), a rate of change of the articulation of the articulated wrist exceeds a configurable angular speed threshold (e.g., 10 to 60 degrees/second), and/or the like. When dithering is to be performed, it is performed using a process 430. In some examples, when the instrument command is to change the amount of force to be exerted by the jaws, dithering should be performed when the instrument command results in one or more grasping conditions being present. In some examples, the one or more grasping conditions include whether the amount of force applied by the jaws to the grasped material exceeds a configurable force threshold (e.g., 40-50 Newtons), the amount of force applied by the jaws to the grasped material is below a configurable force threshold (e.g., 5 to 10 Newtons), a change in the amount of force applied by the jaws exceeds a configurable force change threshold (e.g., 8 to 18 Newtons), and/or the like. In some examples, the amount of force applied by the jaws may be determined using one or more sensors (e.g., one or more pressure sensors, strain gauges, and/or the like) located on the jaws or drive train 250. When dithering is to be performed, it is performed using a process 430. When dithering is not to be performed, the instrument command is performed without dithering using a process 440.

At the process 430, one or more actuator control signals are dithered. According to some embodiments, the one or more actuator control signals are dithered based on four configurable parameters: a signal shape or the dithering, a magnitude to the dithering, a frequency of the dithering, and a duration of the dithering. In some examples, the signal shape of the dithering may be a sinusoid, a square wave, a triangular wave, a sawtooth wave, and/or the like. In some examples, the dithering may be a combination of multiple signal shapes (e.g., components at two or more frequencies, a square wave with sinusoidal ripples, and/or the like).

In some examples, the magnitude of the dithering may be a configurable percentage (e.g., 10-30 percent) of a setpoint for a respective actuator control signal. In some examples, the setpoint may correspond to a position setpoint of the actuator (e.g., a position where it is driving the jaws of the end effector), a force and/or torque setpoint (e.g., an amount of force or torque that may be applied by the respective actuator to actuate the jaws), a current setpoint for current supplied to the respective actuator (e.g., which is typically related to applied force and/or torque), a combination of some of all of the above, and/or the like. In some examples, the magnitude of the dithering may be a configurable percentage of a setpoint limit (e.g., a position, force, torque, current limit and/or the like) for the respective actuator. In some examples, the amount of dithering may be limited so as not to exceed a setpoint limit of the respective actuator and/or so that a configurable minimum grasp force is maintained. In some examples, although dithering might be configured around a setpoint like torque, limits on position and/or velocity may additionally be enforced that may cause clipping of the dithering signal for certain use cases. In some examples, the limiting of the amount of dithering may result in asymmetric dithering about the setpoint for the respective actuator. In some examples, the magnitude of the dithering may be inversely related to a duration of the dithering.

In some examples, the frequency of the dithering may be at a configurable frequency (e.g., 5-10 Hertz). In some examples, the frequency of the dithering may be at or slightly above (e.g., 1-5 Hertz) above a natural frequency for the instrument due to contributions by the respective actuator, the drive unit (e.g., drive system 240), the one or more drive mechanisms (e.g., the one or more drive mechanisms 250), effects of the articulation of the articulated wrist, the jaws, and/or the grasped material. In some examples, the natural frequency may be determined empirically, such as during calibration of the instrument. In some examples, the frequency may be additionally adjusted as a function of dithering duration and/or number of dithering cycles such that the frequency increases or decreases with the completion of each cycle and/or over time.

In some examples, the duration of the dithering may depend on the type of the instrument command received during process 410. In some examples, when the instrument command is to apply cutting and/or sealing energy, the dithering may be applied for a first configurable period of time (e.g., 0.25-1.0 seconds) before the energy is applied, such that the application of the cutting and/or sealing energy is delayed. In some examples, the dithering is applied throughout the application of the cutting and/or sealing energy. In some examples, the dithering is continued after the application of the cutting and/or sealing energy ends for a second configurable period of time (e.g., 0.25-1.0 seconds). In some examples, when the instrument is commanded to change the articulation of the articulated wrist, the dithering may begin when the articulation condition that triggered dithering (e.g., amount of articulation, change in articulation, and/or angular speed) is detected and continues until further articulation stops. In some examples, the articulation may be considered stopped when a change in articulation command for the articulated wrist is no longer being received, an angular speed of the articulation is below a configurable threshold, the articulation condition is no longer present, and/or the like. In some examples, the articulation may be considered stopped when the change in articulation command for the articulated wrist is no longer being received, an angular speed of the articulation is below the configurable threshold, the articulation condition is no longer present, and/or the like for a configurable period of time (e.g., 0.5-1.0 seconds).

In some examples, when the instrument is commanded to change the amount of force to be applied by the jaws, the dithering may begin when the grasping condition that triggered dithering (e.g., amount of force applied by the jaws or change in the amount of force applied by the jaws) is detected and continues until further change in the amount of force applied by the jaws stops. In some examples, the change in the amount of force applied by the jaws may be considered stopped when a change in force to apply command for the jaws is no longer being received, an amount of a change in the force applied by the jaws is below a configurable threshold, the grasping condition is no longer present, and/or the like. In some examples, the change in the amount of force applied by the jaws may be considered stopped when the change in force to apply command for the jaws is no longer being received, the amount of the change in the force applied by the jaws is below the configurable threshold, the grasping condition is no longer present, and/or the like for a configurable period of time (e.g., 0.5-1.0 seconds).

In some examples, the time periods may be monitored and/or tracked using one or more programmable timers. In some examples, the duration of the dithering may be inversely related to the magnitude of the dithering.

According to some embodiments, the dithering may be implemented in the one or more actuator control signals by applying the dithering to a setpoint of the respective actuator. In some examples, the dithering may be applied to the setpoint by superimposing it on the setpoint. In some examples, a nominal value for the setpoint may be determined from the instrument command received during process 410. In some examples, the setpoint may be the setpoint used by the controller of the respective actuator to control a force and/or torque exerted by the respective actuator. In some examples, the setpoint may be a force setpoint, a torque setpoint, a current setpoint, and/or the like. In some examples, the setpoint may be a setpoint used by the controller for the respective actuator to control a position of the respective actuator that controls a position of one or more of the jaws, a separation between the jaw, and/or the like.

At the process 440, the instrument command is performed. Depending upon whether process 430 was performed the instrument command is performed with or without dithering. In some examples, the instrument command is performed by setting one or more setpoints (e.g., the nominal setpoint described with respect to process 430) for a controller used to control the instrument and/or the end effector. In some examples, the instrument command may result in one or more of a change in the articulation of the articulated wrist, a change in separation in the jaws, a change in force applied to the grasped material by the jaws, application of cutting and/or sealing energy to the material by the one or more electrodes, and/or the like. In some examples, the instrument command may be performed by sending one or more actuator signals to the respective actuators, sending one or more signals to a controller for energy delivery, and/or the like.

Once the instrument command is performed, method 400 may be repeated for another instrument command by returning to process 410.

As discussed above and further emphasized here, FIG. 4 is merely an example which should not unduly limit the scope of the claims. One of ordinary skill in the art would recognize many variations, alternatives, and modifications. According to some embodiments, the various properties (e.g., thresholds, percentages, time periods, and/or the like) of method 400 may be configured based on one or more factors and/or combination of the one or more factors. In some examples, the one or more factors include a type of the instrument, calibration of the instrument, an age of the instrument (e.g., a number of previous uses), a procedure being performed, operator preference, a type of the material being grasped, a separation (e.g., angle and/or distance) between the jaws, a rate of change in separation between the jaws, an amount of articulation of the articulated wrist, an angular speed of the change in articulation of the articulated wrist, and/or the like. In some examples, an amount of dithering (e.g., magnitude and/or duration) may be higher for an instrument that has been used fewer times. In some examples, an amount of dithering may be higher for larger jaw separations, larger rates of change in the separation of the jaws, a higher amount of articulation of the articulated wrist, a larger angular speed of the change in the articulation of the articulated wrist, an amount of force applied by the jaws to a grasped material and/or the like. In some examples, the amount of force applied by the jaws to the grasped material may be determined using one or more sensors (e.g., one or more pressure sensors, strain gauges, and/or the like) located on the jaws and/or the drive mechanisms.

Some examples of control units, such as control unit 140 may include non-transitory, tangible, machine-readable media that include machine-readable instructions that when run by one or more processors (e.g., processor 150) may cause the one or more processors to perform the processes of method 400. Some common forms of machine readable media that may include the processes of method 400 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and in a manner consistent with the scope of the embodiments disclosed herein.

1. A computer-assisted device comprising:
an instrument having a first jaw and a second jaw configured to grasp a material;
one or more actuators configured to actuate the first and second jaws to apply force to the grasped material; and
a controller coupled to the one or more actuators;
wherein the controller is configured to:
determine that actuation of the one or more actuators should be dithered; and
in response to the determination, dither one or more control signals to the one or more actuators so as to cause variations in a force or torque applied by the one or more actuators.

2. The computer-assisted device of claim 1, wherein the material is bodily tissue.

3. The computer-assisted device of claim 1, wherein the one or more control signals correspond to a force setpoint for the one or more actuators, a torque setpoint for the one or more actuators, or a current setpoint for the one or more actuators.

4. (canceled)

5. The computer-assisted device of claim 1, wherein the dithering is superimposed on the one or more control signals.

6. (canceled)

7. The computer-assisted device of claim 1, wherein a magnitude of the dithering is:
a percentage of a setpoint for a respective one of the one or more control signals; or
a percentage of a setpoint limit for a respective one of the one or more control signals.

8. (canceled)

9. The computer-assisted device of claim 1, wherein a magnitude of the dithering is limited so as not to violate one or more setpoint limits for a respective one of the one or more control signals.

10. (canceled)

11. The computer-assisted device of claim 1, wherein a frequency of the dithering is between 5 and 10 Hertz inclusive.

12. The computer-assisted device of claim 1, wherein a frequency of the dithering is at or above a natural frequency for the instrument.

13. The computer-assisted device of claim 1, wherein the controller is further configured to determine one or more properties of the dithering based on one or more of:
a type of the instrument;
a calibration of the instrument;
a number of previous uses of the instrument;
a procedure being performed with the instrument;
operator preference;
a type of the material;
a separation between the first and second jaws;
a change in the separation between the first and second jaws;
an articulation of an articulated wrist of the instrument;
an angular speed of the articulation; or
an amount of the force applied to grasp the material.

14-15. (canceled)

16. The computer-assisted device of claim 1, wherein the controller is further configured to perform at least one of:
begin dithering of the one or more control signals for a first period of time before cutting energy, sealing energy, or both cutting and sealing energy is applied using one or more electrodes on the first jaw, the second jaw, or both the first jaw and the second jaw; or
continue the dithering of the one or more control signals for a second period of time after the cutting energy, the sealing energy, or both the cutting and sealing energy is no longer applied.

17-18. (canceled)

19. The computer-assisted device of claim 1, wherein to determine that the actuation of the one or more actuators should be dithered, the controller is configured to determine whether one or more articulation conditions is present, the one or more articulation conditions including:
an articulation of an articulated wrist of the instrument is above an articulation threshold;
a change in the articulation of the articulated wrist is above a change in articulation threshold; or
a rate of change of the articulation of the articulated wrist is above an angular speed threshold.

20-21. (canceled)

22. The computer-assisted device of claim 1, wherein to determine that the actuation of the one or more actuators should be dithered, the controller is configured to determine whether one or more grasping conditions is present, the one or more grasping conditions including:
the force applied to grasp the material by the first and second jaws is below a first force threshold;
the force applied to grasp the material by the first and second jaws is above a second force threshold; or
a change in the force applied to grasp the material by the first and second jaws is above a force change threshold.

23-27. (canceled)

28. A method comprising:
determining, by a controller of a computer-assisted device, that actuation of one or more actuators of the computer-assisted device should be dithered, the one or more actuators usable to actuate a first jaw and a second jaw of an instrument to grasp a material; and
in response to the determining, dithering, by the controller, one or more control signals to the one or more actuators so as to cause variations in a force or torque applied by the one or more actuators.

29. (canceled)

30. The method of claim 28, wherein the one or more control signals corresponds to a force setpoint for the one or more actuators, a torque setpoint for the one or more actuators, or a current setpoint for the one or more actuators.

31-33. (canceled)

34. The method of claim 28, wherein a magnitude of the dithering is:
a percentage of a setpoint for a respective one of the one or more control signals;
a percentage of a setpoint limit for a respective one of the one or more control signals; or
limited so as not to violate one or more setpoint limits for a respective one of the one or more control signals.

35-39. (canceled)

40. The method of claim 28, further comprising determining one or more properties of the dithering based on one or more of:
a type of the instrument;
a calibration of the instrument;
a number of previous uses of the instrument;
a procedure being performed with the instrument;
operator preference;
a type of the material;
a separation between the first and second jaws;
a change in the separation between the first and second jaws;
an articulation of an articulated wrist of the instrument;
an angular speed of the articulation; or
an amount of the force applied to grasp the material.

41-42. (canceled)

43. The method of claim 28, wherein the method further comprises one or more of:
beginning dithering of the one or more control signals for a first period of time before cutting energy, sealing energy, or both cutting and sealing energy is applied using one or more electrodes on the first jaw, the second jaw, or both the first jaw and the second jaw; or
continuing the dithering of the one or more control signals for a second period of time after the cutting energy, the sealing energy, or both the cutting and sealing energy is no longer applied.

44-54. (canceled)

55. A non-transitory machine-readable medium comprising a plurality of machine-readable instructions which when executed by one or more processors associated with a computer-assisted device are adapted to cause the one or more processors to perform a method comprising:
determining that actuation of one or more actuators of the computer-assisted device should be dithered, the one or more actuators usable to actuate a first jaw and a second jaw of an instrument to grasp a material; and
in response to the determining, dithering one or more control signals to the one or more actuators so as to cause variations in a force or torque applied by the one or more actuators.

56. The non-transitory machine-readable medium of claim 55, wherein the one or more control signals corresponds to a force setpoint for the one or more actuators, a torque setpoint for the one or more actuators, or a current setpoint for the one or more actuators.

57. The non-transitory machine-readable medium of claim 55, wherein the method further comprises one or more of:
beginning dithering of the one or more control signals for a first period of time before cutting energy, sealing energy, or both cutting and sealing energy is applied using one or more electrodes on the first jaw, the second jaw, or both the first jaw and the second jaw; or
continuing the dithering of the one or more control signals for a second period of time after the cutting energy, the sealing energy, or both the cutting and sealing energy is no longer applied.
